(19) United States
(12) Patent Application Publication
(10) Pub. No.: US 2016/0201070 A1
(43) Pub. Date: Jul. 14, 2016

(54) Method for Expanding Color Palette in Dendrobium Orchids

(71) Applicant: University of Dubuque, Dubuque, IA (US)

(72) Inventor: Rasika G. Mudalige-Jayawickrama, Dubuque, IA (US)

(21) Appl. No.: 15/011,225

(22) Filed: Jan. 29, 2016

Related U.S. Application Data
(63) Continuation-in-part of application No. 13/946,948, filed on Jul. 19, 2013, now abandoned.
(60) Provisional application No. 61/674,287, filed on Jul. 20, 2012.

(51) Int. Cl. C12N 15/82

(52) U.S. Cl. CPC C12N 15/825; C12N 15/8218

(57) ABSTRACT

A nucleotide sequence encoding flavonoid 3′-hydroxylase (F3′H) of Dendrobium, a method of producing a transgenic flower color-changed Dendrobium plant, and a transgenic flower color-changed Dendrobium plant are provided by this invention.

This application is a Continuation-in-Part Application of U.S. Patent application Ser. No. 13/946,948, which claims priority to U.S. Provisional Patent Application No. 61/674,287 filed Jul. 20, 2012, the entire contents of each of which are incorporated herein by reference.

### BACKGROUND OF THE INVENTION

1. Field of the Invention

The invention disclosed herein relates generally to the use of recombinant DNA technology to produce Dendrobium orchids having orange (pelargonidin-accumulating) and blue (delphinidin-accumulating) flowers. Particularly, the invention provides methods for modifying anthocyanin biosynthesis in Dendrobium orchids through gene suppression.

2. Description of Related Art

Dendrobium, a member of the Orchidaceae family, is one of the largest living genera with approximately 1400 species and many man-made hybrids. Classical breeding techniques have given rise to many commercially successful hybrids with attractive flower colors and forms, long vase life, fragrance, seasonality, and desirable spray length.

However, most commercial Dendrobium hybrids display predominantly purple, lavender, or pink flower colors due to cyanidin and peonidin accumulation. A chemical survey of commercial Dendrobium hybrids has shown that some colors such as orange-red and blue are missing from Dendrobium flower color spectrum (Kuehnle et al., 1997, Euphytica 95: 187-194).

Unlike moth orchids and Cymbidium, where the lack of a blue flower color is likely due to weak expression of flavonoid 3′,5′-hydroxylase (F3′5′H) (see U.S. Patent Application Publication No. 2011/0191907), the limited color range within Dendrobium species can be due to the absence, mutation, or over-activity of an anthocyanin biosynthetic gene. (Johnson et al., 1999, Plant J. 19:81-5).

Although substrate specificity of dihydroflavonol 4-reductase (DFR) may explain the absence of certain colors among some ornamental plants, Obsuwan et al., 2007, Acta Hort. (ISHS) 764:137-44 has shown that Dendrobium DFR can efficiently catalyze reduction of dihydrokaempferol (DHK), dihydroquercetin (DHQ), and dihydromyricetin (DHM), resulting in the production of pelargonidin, cyanidin and delphinidin with no substrate specificity.

DFR substrate specificity in orchids has been previously investigated. For example, DFR from Petunia and Cymbidium orchid cannot reduce DHK efficiently, explaining the lack of pelargonidin-accumulating orange flowers even in the absence of competing enzymes flavonoid 3′-hydroxylase (F3′H) and F3′5′H (Forkmann & Ruhnau, 1987, Z. Naturforsch. 42c: 1146-8; Gerats et al., 1982, Planta 155:364-8; Johnson et al., 1999, Plant J. 19:81-5).

Johnson et al., 2001, J. Biol. Chem. 276:172-8) has demonstrated that substrate specificity is found in DFR from Cymbidium orchid by heterologous expression in a Petunia host. Substrate specificity was not, however, found in Dendrobium DFR inside a similar Petunia host (Mudalige-Jayawickrama et al., 2005, J. Amer. Soc. Hort. Sci. 130:611-8; Obsuwan et al., 2007, Id.). Therefore, the rarity of pelargonidin-accumulating flowers in Dendrobium may be due to the competition from a robust F3′H enzyme that siphons off a necessary intermediate DHK into purple pathway. (Mudalige-Jayawickrama et al., 2005, Id.).

Thus, there is a need in the art to delineate the biochemical basis of Dendrobium flower color by isolating and characterizing anthocyanin biosynthetic genes, particularly the gene encoding F3′H, in order to determine the basis for lack of blue delphinidin and rarity of orange pelargonidin among commercial Dendrobium hybrids. There is also a commercial need to produce Dendrobium orchids with modified flower colors, including rare colors, such as orange or blue.

### SUMMARY OF THE INVENTION

It is against the above background that the present invention provides certain advantages and advancements over the prior art.

Although this invention as disclosed herein is not limited to specific advantages or functionalities, the invention provides method for producing a transgenic plant, comprising:
(a) transfecting a plant with a genetic construct comprising an antisense suppressor of a nucleic acid molecule having at least 90% identity to a nucleotide sequence set forth in SEQ ID NO:1; and
(b) expressing the genetic construct in cells of the plant.

In some aspects of the method for producing a transgenic plant disclosed herein, the antisense suppressor comprises an antisense suppressor having at least 90% identity to a sequence set forth in any one of SEQ ID NOs: 93-96.

In some aspects of the method disclosed herein, the genetic construct is expressed in the protocorm-like bodies of the plant.

In some aspects of the method disclosed herein, the transgenic plant is a flower color-changed plant and wherein the plant is a native-color plant.

In some aspects, the flower color-changed plant and the native-color plant are of the Orchidaceae family.

In some aspects, the flower color-changed plant and the native-color plant are Dendrobium orchids.

The invention further provides method for producing a flower color-changed plant having an orange flower, comprising:
(a) transfecting a native-color plant having a purple flower with a genetic construct comprising an antisense suppressor of a nucleic acid molecule having at least 90% identity to a nucleotide sequence set forth in SEQ ID NO:1; and
(b) expressing the genetic construct in cells of the plant.

In some aspects of the method for producing a flower color-changed plant having an orange flower disclosed herein, the antisense suppressor comprises an antisense suppressor having at least 90% identity to a sequence set forth in any one of SEQ ID NOs:93-96.

In some aspects of the method disclosed herein, the genetic construct is expressed in the protocorm-like bodies of the plant.

In some aspects of the method disclosed herein, the flower color-changed plant and the native-color plant are of the Orchidaceae family.

In some aspects, the flower color-changed plant and the native-color plant are Dendrobium orchids.

The invention further provides a flower color-changed plant produced by a method disclosed herein.

In some aspects, the flower color-changed plant is of the Orchidaceae family.

In some aspects, the flower color-changed plant is a Dendrobium orchid.

The invention further provides a flower color-changed plant comprising in cells thereof a genetic construct comprising an antisense suppressor of a nucleic acid molecule having at least 90% identity to a nucleotide sequence set forth in SEQ ID NO:1.

In some aspects, the antisense suppressor comprises an antisense suppressor having at least 90% identity to a sequence set forth in any one of SEQ ID NOs:93-96.

In some aspects, the flower color-changed plant is of the Orchidaceae family.

In some aspects, the flower color-changed plant is a Dendrobium orchid.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The patent or patent application file contains at least one drawing executed in color. Copies of this patent or patent application publication with color drawing(s) will be provided by the Office upon request and payment of the necessary fee.

The following detailed description of the embodiments of the present invention can be best understood when read in conjunction with the following drawings, where like structure is indicated with like reference numerals and in which:

FIG. 1 shows that flavonoids are synthesized via a complex biochemical pathway known as the phenylpropanoid pathway. (A) Typical purple Dendrobium hybrid. (B) Rare pelargonidin accumulating mutant. (C) Anthocyanin biosynthetic pathway with the enzyme abbreviations. Dihydrokaempfeol (DHK) intermediate is surrounded by the red circle.

FIG. 2 shows chemical analysis of the purple Dendrobium flower UH503 and the Petunia W80 mutant flowers transformed with 35S: Antirrhinum Dfr and 35S:—Dendrobium Dfr. The pelargonidin and orange color in Den-Dfr transformant. (Obsuwan et al., 2007, Id.).

FIG. 3A shows multiple sequence alignments of deduced amino acid sequences of Dendrobium—F3′H and other plant species using CLUSTALW. The “*” represent conserved amino acids; the “:” represents similar amino acids substitutions. Dendrobium_Jaquelyn_Thomas (SEQ ID NO:12); Lilium hybrid (SEQ ID NO:13); Sorghum bicolor (SEQ ID NO:14); Zea mays (SEQ ID NO:15); Allium cepa (SEQ ID NO:16); Antirrhinum majus (SEQ ID NO:17); Torenia hybrid (SEQ ID NO:18); Malus_x_domestica (SEQ ID NO:19); Matthiola incana (SEQ ID NO:20); Pelargonium×hortorum (SEQ ID NO:21). FIG. 3B shows phylogenetic relationships determined by amino acid sequence similarity (PHYLIP version 3.5c).

FIG. 4 shows photographs of agarose gel electrophopretic analyses of RT-PCR products of F3′H and DFR in different floral organs of D. Jaquelyn Thomas ‘Uniwai Prince’ (UHSO3) and D. Icy Pink ‘Sakura’ (K1224) orchids. Different stages of floral buds used for analysis are shown on top.

FIG. 5 shows a schematic representation of different strategies that can be used to increase the color pallete of commercial Dendrobium hybrids.

FIG. 6 shows the annealing regions of ARO793 (SEQ ID NO:22), ARO1190 (SEQ ID NO:23), ARO958 (SEQ ID NO:24), ARO1342 (SEQ ID NO:25), ARO1381 (SEQ ID NO:26), and ARO1485 (SEQ ID NO:27) to the Dendrobium F3′H sequence (SEQ ID NO:1).

FIG. 7A shows direct delivery of antisense RNA oligonucleotides (ARO) via the cut end of a Dendrobium inflorescence. FIG. 7B shows a method of direct injection of an ARO using a sterile needle. FIG. 7C shows surface-sterilized flower buds placed in MS media, wherein an ARO solution was placed into a cut hole in the media. FIG. 7D shows the cut end of a Dendrobium bud inserted into a microfuge tube comprising an ARO solution.

FIG. 8 shows agarose gels analyzing RT-PCR products of F3′H mRNA before and after direct bud feeding through the pedicel.

Skilled artisans will appreciate that elements in the figures are illustrated for simplicity and clarity and have not necessarily been drawn to scale. For example, the dimensions of some of the elements in the figures can be exaggerated relative to other elements to help improve understanding of the embodiment(s) of the present invention.

### DETAILED DESCRIPTION OF THE INVENTION

All publications, patents and patent applications cited herein are hereby expressly incorporated by reference for all purposes.

Before describing the present invention in detail, a number of terms will be defined. As used herein, the singular forms “a,” “an,” and “the” include plural referents unless the context clearly dictates otherwise. For example, reference to a “nucleic acid” means one or more nucleic acids.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Methods well known to those skilled in the art can be used to construct genetic expression constructs and recombinant cells according to this invention. These methods include in vitro recombinant DNA techniques, synthetic techniques, in vivo recombination techniques, and polymerase chain reaction (PCR) techniques. See, for example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wley Interscience, New York, and PCR Protocols: A Guide to Methods and Applications (Innis et al., 1990, Academic Press, San Diego, Calif.).

As used herein, the terms “polynucleotide,” “nucleotide,” “oligonucleotide,” and “nucleic acid” can be used interchangeably to refer to nucleic acid comprising DNA, RNA, derivatives thereof, or combinations thereof, in either single-stranded or double-stranded embodiments depending on context as understood by the skilled worker.

As used herein, the term “recombinant host” is intended to refer to a host, the genome of which has been augmented by at least one DNA sequence. Such DNA sequences include but are not limited to genes that are not naturally present, DNA sequences that are not normally transcribed into RNA or translated into a protein (“expressed”), and other genes or DNA sequences which one desires to introduce into a host. It will be appreciated that typically the genome of a recombinant host described herein is augmented through stable introduction of one or more recombinant genes. Generally, introduced DNA is not originally resident in the host that is the recipient of the DNA, but it is within the scope of this disclosure to isolate a DNA segment from a given host, and to subsequently introduce one or more additional copies of that DNA into the same host, e.g., to enhance production of the product of a gene or alter the expression pattern of a gene. In some instances, the introduced DNA will modify or even replace an endogenous gene or DNA sequence by, e.g., homologous recombination or site-directed mutagenesis. Suitable recombinant hosts include plants and plant cells.

As used herein, the term “recombinant gene” refers to a gene or DNA sequence that is introduced into a recipient host, regardless of whether the same or a similar gene or DNA sequence may already be present in such a host. “Introduced,” or “augmented” in this context, is known in the art to mean introduced or augmented by the hand of man. Thus, a recombinant gene can be a DNA sequence from another species or can be a DNA sequence that originated from or is present in the same species but has been incorporated into a host by recombinant methods to form a recombinant host. It will be appreciated that a recombinant gene that is introduced into a host can be identical to a DNA sequence that is normally present in the host being transformed, and is introduced to provide one or more additional copies of the DNA to thereby permit overexpression or modified expression of the gene product of that DNA. In some aspects, said recombinant genes are encoded by cDNA.

As used herein, the term “engineered biosynthetic pathway” refers to a biosynthetic pathway that occurs in a recombinant host, as described herein. In some aspects, one or more steps of the biosynthetic pathway do not naturally occur in an unmodified host. In some embodiments, a heterologous version of a gene is introduced into a host that comprises an endogenous version of the gene.

As used herein, the term “endogenous” gene refers to a gene that originates from and is produced or synthesized within a particular organism, tissue, or cell. In some embodiments, the endogenous gene is a plant gene. In some embodiments, the gene is endogenous to Dendrobium. As used herein, the term “overexpress” is used to refer to the expression of a gene in an organism at levels higher than the level of gene expression in a wild type organism. See, e.g., Prelich, 2012, Genetics 190:841-54. In some embodiments, an endogenous gene is deleted. As used herein, the terms “deletion,” “deleted,” “knockout,” “knocked out,” “shut down,” “silenced,” and “silencing” can be used interchangabley to refer to an endogenous gene that has been manipulated to no longer be expressed in an organism, including, but not limited to, Dendrobium.

As used herein, the terms “heterologous sequence” and “heterologous coding sequence” are used to describe a sequence derived from a species other than the recombinant host. In some embodiments, the recombinant host is a plant, such as a Dendrobium orchid, and a heterologous sequence is derived from an organism other than a Dendrobium orchid. A heterologous coding sequence, for example, can be from a prokaryotic microorganism, a eukaryotic microorganism, a plant, an animal, an insect, or a fungus different than the recombinant host expressing the heterologous sequence. In some embodiments, a coding sequence is a sequence that is native to the host.

As used herein, the term “transgenic plant” refers to a plant that has been genetically engineered to comprise a characteristic other than the characteristics of a native plant. For example, the characteristic can be a physical characteristic such as color, size, or shape.

As used herein, the terms “antisense suppressor,” “antisense oligonucleotide,” “antisense construct,” “antisense RNA oligonucleotide (ARO),” “artificial microRNA (amiRNA),” “ARO molecule,” and “ARO construct” can be used interchangeably to refer to an oligonucleotide that has been engineered to shut down expression of one or more target genes. See Dias & Stein, 2002, Mol Cancer Ther 1(5):347-55. As used herein, the term “target gene” can be used to refer to a gene to be silenced. As used herein, ARO constructs are single-stranded, comprise 21 nucleotides, and anneal to a particular region of the target gene. Deliver of an ARO molecule to a plant or plant cell can result in permanent silencing of a target gene. See Roberts, 2005, Plant Methods 1:12; Schwab et al., Plant Cell. 18(5):1121-33; Sun et al., 2005, Plant J. 44:128-38; Unnamalai et al., 2004, FEBS Lett. 566:307-10; and Ossowski et al., 2008, Plant J. 53(4):674-90. In some aspects, direct delivery of an ARO molecule to a plant or plant cell can be less expensive, labor intensive, and/or time consuming than delivery of a small RNA molecule by vector based methods of transient or stable expression.

As used herein, the term “anthocyanidin” is used to refer to a water-soluble pigment (colored flavonoid glycoside) that accumulates in a plant cell vacuole, giving characteristic colors to flowers and fruits and can be responsible for red-pink cyanidin, orange pelargonidin, and blue delphinidin in flowers. Production of the three primary classes of anthocyanidins by the phenyl propanoid pathway is controlled by the availability of the colorless substrates DHK, DHQ, and DHM and the activities of F3′H, F3'S′H, and DFR. Conversion of those three dihydroflavonoids into leucoanthocyanidins is a required step in anthocyanin biosynthesis and is catalyzed by DFR.

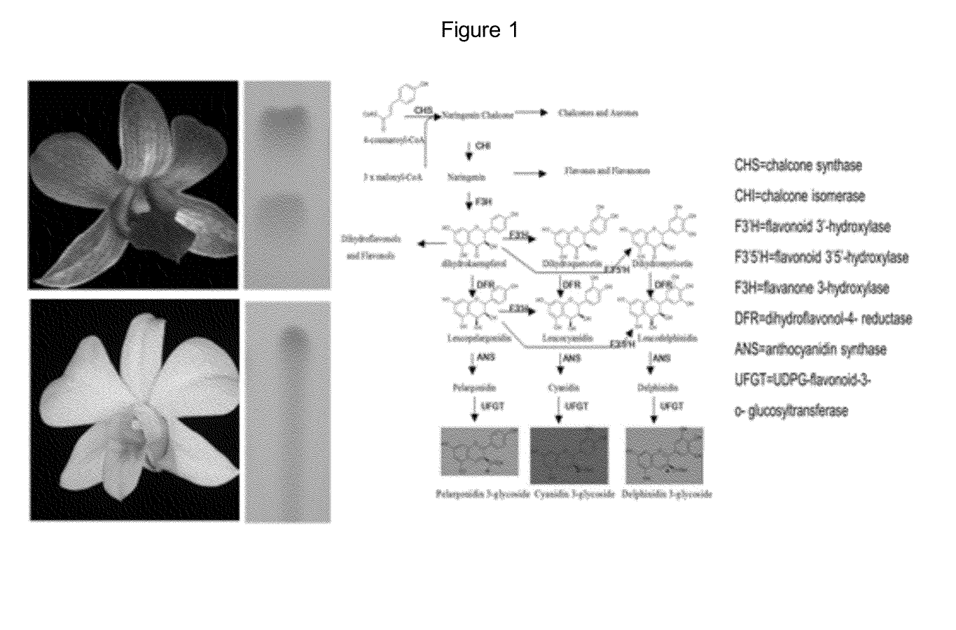

Dendrobium, the largest genus of the orchid family, display predominantly, purple, lavender and pink flowers due to cyanidin and peonidin accumulation (FIG. 1). Blue delphinidin is absent in Dendrobium hybrids, while orange pelargonidin (FIG. 1) is found in a few rare mutants (FIG. 1; Kuehnle et al., 1997, Id.).

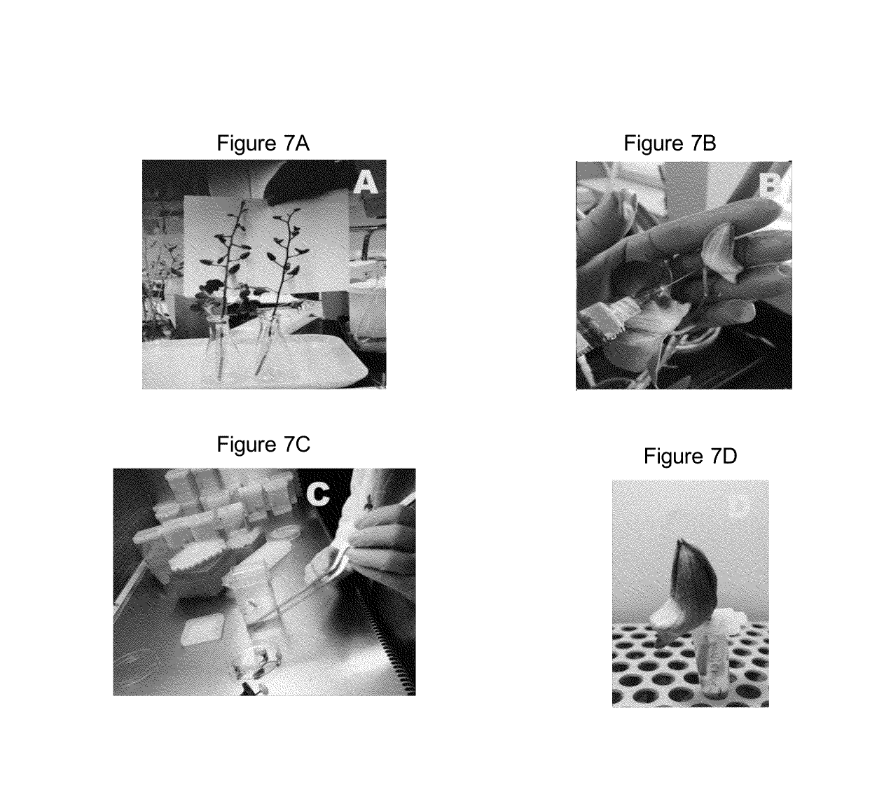

As used herein, the term “influorescence” refers to the complete flower head of a plant, including stems, stalks, bracts, flower buds, and flowers. The term “pedicel” refers to a stem that attaches a flower to an influorescence. The term “petiole” refers to a stalk that attaches a leaf blade to a stem. The term “protocorm” is used to refer to tuber shaped undifferentiated young seedlings. The terms “protocorm-like-bodies” and “PLBs” are used to refer to undifferentiated tissues with multiple meristems. See Example 4 and FIG. 7.

DFR is important in flower color due to its substrate specificity. Substrate specificity of DFR explains the absence of certain colors among some ornamental plants, which makes DFR an important target for flower color manipulation through genetic engineering. In order to characterize DFR in two major subtropical orchids, full-length cDNA clones encoding DFR are isolated using a RT-PCR based technique from petals of hybrid plants resulting from Dendrobium×Icy Pink ‘Sakura’ and Oncidium×Gower Ramsey genetic crosses.

The substrate specificity of Dendrobium DFR and Oncidium DFR were investigated by genetic transformation of the mutant Petunia line W80 that predominantly accumulates DHK. Chemical analysis of transformed lines revealed that both Dendrobium DFR and Oncidium DFR can efficiently catalyze the reduction of DHK, DHQ, and DHM and can result in the production of pelargonidin, cyanidin, and delphinidin with no substrate specificity.

In order to understand the reason for lack of blue delphinidin and rarity of orange pelargonidin among commercial Dendrobium hybrids, the biochemical basis of Dendrobium flower color was delineated as set forth herein by isolation and characterization of certain anthocyanin biosynthetic genes. As a consequence, disclosed herein are methods for expanding the available flower colors for Dendrobium and other orchids through genetic manipulation.

In orchids, flavonoids are synthesized via a complex biochemical pathway known as the phenylpropanoid pathway (FIG. 1). The first committed step of flavonoid biosynthesis is condensation of 3 molecules of malonyl-CoA with a single molecule of 4-coumaroyl-CoA to form chalcone, catalyzed by the enzyme chalcone synthase (CHS). Chalcone is then isomerized to naringenin, a colorless flavonone, by chalcone isomerase (CHI). Naringenin is subsequently hydroxylated by flavanone 3-hydroxylase (F3H) to form DHK, a common intermediate to several flavonoid species. DHK can be hydroxylated at the 3′ position of the B ring to form DHQ or at both the 3′ and 5′ positions to form DHM; the DHQ reaction is catalyzed by F3′H and the DHM reaction is catalyzed by F3′5′H. DHK is an intermediate that can be utilized by all three branches of the pathway to produce orange pelargonidin, purple cyanidin or blue delphinidin as the final anthocyanidin. Dihydroflavonol 4-reductase can accept DHK, DHQ or DHM to produce orange, purple and blue colors, respectively.

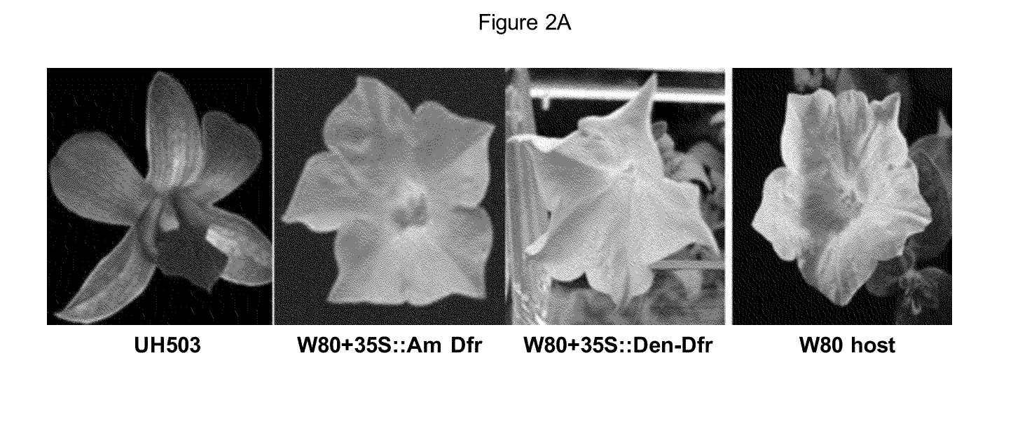

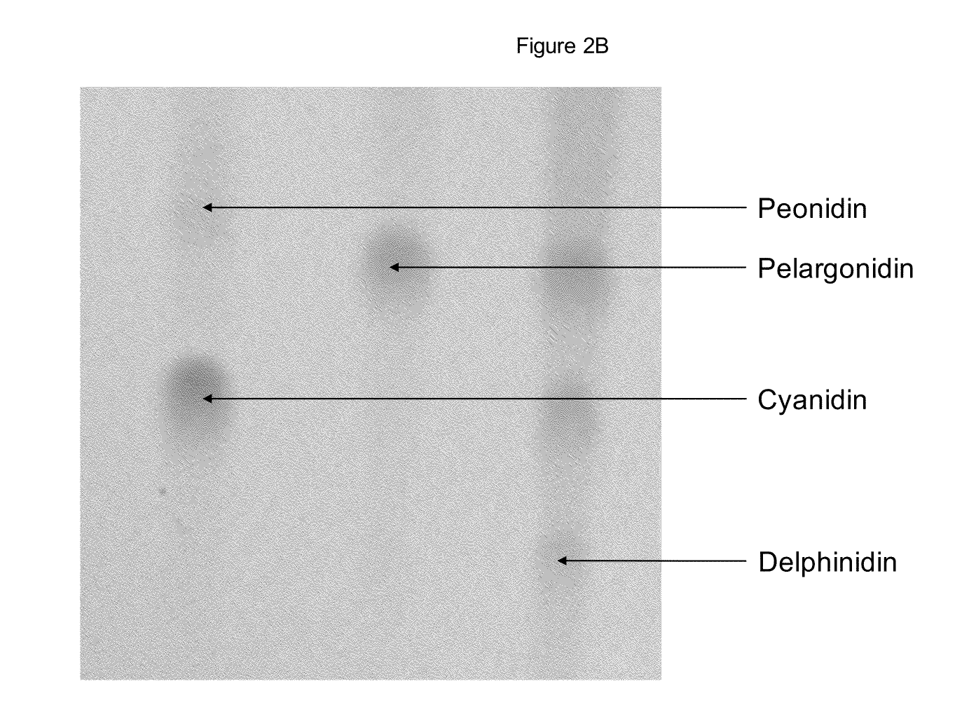

Substrate specificity of DFR was investigated through heterologous expression of Dendrobium DFR in a Petunia host. Petunia DFR cannot efficiently reduce DHK to produce orange pelargonidin-accumulating flowers, even in the absence of competing enzymes F3′H and F3′5′H (FIG. 2; W80). Zea mays DFR enzyme efficiently catalyzed the reduction of DHK to produce novel transgenic orange colored Petunia (Meyer et al., 1987, Nature 330: 677-8). However, Orchid DFR enzymes produced contradicting results when inserted into the same Petunia host. The Cymbidium orchid DFR did not reduce DHK to make pelargonidin efficiently (Johnson et al., 1999, Id.) whereas Dendrobium DFR was able to make orange pelargonidin (FIG. 2; Obsuwan et al., 2007, Id.). In some aspects, Petunia leaf discs were transformed with DFR constructs using Agrobacterium mediated transformation (Obsuwan et al. 2007, Id.). Dendrobium Icy Pink ‘Sakura’ PLBs were transformed with UBQ3:Antirrhinum DFR via Biolistic bombardment (BIO-RAD).

Dendrobium DFR is capable of accepting the precursors of all three colors, orange, purple, and blue in Petunia. Therefore, substrate specificity of DFR does not determine the flower color of Dendrobium and is not the basis for predominance of purple color in Dendrobium hybrids. Without wishing to be bound by a theory, it is believed that enzyme competition among DFR, F3′H, and F3′5′H determines flower color of Dendrobium orchid.

The predominance of cyanidin occurs either due to substrate specificity of the DFR enzyme or enzymatic competition among DFR, F3′H, and F3′5′H for the common substrate dihydrokaempferol.

Without wishing to be bound by a theory, it is believed that a reason for the observed color patterns in orchids is that rare pelargonidin flowers are deficient in F3′H, eliminating enzyme competition for DHK so that DHK is catalyzed directly by DFR towards pelargonidin.

Accordingly, in one aspect, the invention provides a gene (SEQ ID NO:1) encoding F3′H from Dendrobium (SEQ ID NO:2). Deduced amino acid sequence of the full gene is approximately only 70% similar to F3′H sequences from other orchid species. F3′H is expressed in all bud stages with the highest expression in mature buds. Expression declines as the flower opens. F3′H is mutated in the orange, pelargonidin-accumulating mutant, suggesting lack of competition from F3′H may lead to novel orange pelargonidin accumulators.

Discovery of Dendrobium F3′H gene permits evaluation of F3′H gene expression and for it to be determined that rare pelargonidin flowers do not exhibit F3′H expression. Moreover, reduction in F3′H activity via gene suppression can be used to produce orange Dendrobium hybrids and breeding materials, as described herein. See Example 4.

Previous results on the Cymbidium orchid have shown that the predominance of purple anthocyanidins, cyaniding and peonidin, is due to substrate specificity of Dihydrofalavonol 4-reductase enzyme (Johnson et al., 1999, Id.). However, substrate specificity is not the biochemical basis for the color patterns shown in naturally occurring Dendrobium orchids (Johnson et al., 1999, Id.).

First, amino acid residues that render substrate specificity to other DFR enzymes, e.g., Petunia, are not shared by the Dendrobium DFR (Mudalige-Jayawickrama et al., 2005, Id.). Second, heterologous expression of Dendrobium DFR in a Petunia mutant resulted in the production of orange pelargonidin in the transgenic line (Obsuwan et al., 2007, Id.). Therefore, the purple predominance in Dendrobium orchids is due to the competition among DFR, F3′H, and F3′5′H to accept the common intermediate, DHK (Mudalige-Jayawickrama et al., 2012, Poster P02047, Annual Meeting of the American Society of Plant Biologists, Austin Tex.).

Unlike predominantly purple Dendrobium orchids, rare orange pelargonidin-accumulating mutants surprisingly and unexpectedly accept DHK due to the absence of strong competition from the F3′H enzyme similar to a pelargonidin accumulating mutant, Dendrobium Icy Pink “Sakura,” which does not express F3′H (Mudalige-Jayawickrama et al., 2012, Id.).

In preferred embodiments, the invention provides methods for rerouting the anthocyanin biosynthetic pathway from purple cyanidin towards orange pelargonidin by inhibiting F3′H enzyme activity in a purple Dendrobium orchid. In certain embodiments, genetic suppression is accomplished by RNA interference (RNAi) (see Bass, 2000, Cell 101:235-8; Carrington, 2000, Nature 408:150-1; and Carrington & Ambrose, 2003, Science 301:336-8). This method does not produce chimeras of transformed and non-transformed sections in a single plant because gene silencing occurs through an RNAi pathway, which allows gene suppression to occur in a systemic manner.

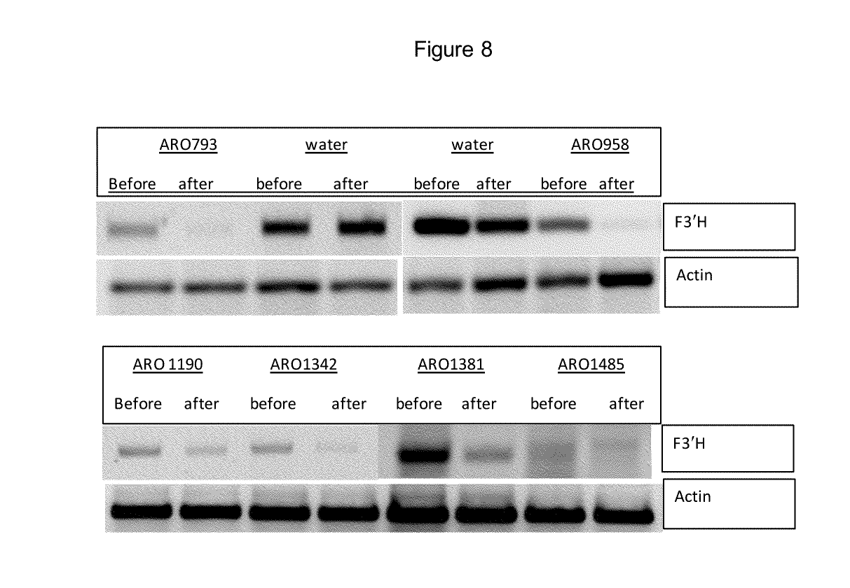

In some embodiments, ARO molecules are designed to shut down the F3′H of a plant or plant cell. In some aspects, the F3′H can be the Dendrobium F3′H of SEQ ID NO:1, SEQ ID NO:2. In some aspects, the Web microRNA Designer (WMD) can be used to design ARO molecules against the F3′H gene. See Schwab et al., Plant Cell. 18(5):1121-33. Non-limiting examples of ARO molecules that can be used to shut down the F3′H gene include ARO793 (SEQ ID NO:22), ARO1190 (SEQ ID NO:23), ARO958 (SEQ ID NO:24), ARO1342 (SEQ ID NO:25), ARO1381 (SEQ ID NO:26), and ARO1485 (SEQ ID NO:27). See Example 4 and FIG. 8. Additional ARO molecules that can be used to shut down the F3′H gene are set forth in SEQ ID NOs:28-91. Suitable ARO constructs can have at least 75% sequence identity to any of the ARO constructs set forth in SEQ ID NOs:22-91. In some aspects, the ARO molecules are methylated at the 5′ and/or 3′ ends. In some apsects, a protein transduction domain (PTD) can be attached to an ARO molecule.

In some embodiments, ARO molecules are delivered to flower buds by i) feeding via a cut end of an inflorescence and/or through a pedicel of an individual bud, ii) transporting in water through a plant's xylem, iii) direct injection into a flower bud via a pedicel, and/or iv) feeding through petiole with ARO-supplemented media. See Example 4.

In some aspects, shutting down of the F3′H gene can reroute anthocyanin biosynthesis towards orange pelargonidin and/or blue delphinidin in an orchid. However, one of skill in the art would recognize that several years are required for orchid propogation. In some embodiments, a sequence is cloned in the antisense orientation and inserted into PLBs of a Dendrobium plant to shut down the F3′H activity. In some aspects, nucleotides 793-1506, nucleotides 958-1506, nucleotides 793-1402, and/or nucleotides 958-1402 of the Dendrobium F3′H gene (SEQ ID NO:1) can be used for construction of ARO constructs to shut down F3′H expression in a Dendrobium plant. Non-limiting examples of antisense constructs to be inserted into PLBs of a Dendrobium plant are set forth in SEQ ID NOs:93-96. In some aspects, a helium gun can be used to generate a transgenic plant, wherein the F3′H gene is shut down. See Mudalige, 2003, “Dendrobium Flower Color: Histology and Genetic Manipulation,” Thesis, University of Hawaii. In some embodiments, transgenic plants are generated using a Particle Inflow Gun, which can be used to deliver gold and/or tungsten particles carrying the gene construct. (Davies, 2013, Methods Mol Biol. 940:63-74; Finer et al., 1992, Plant Cell Reports 11:232-8; Vain et al., 1993, Plant Cell Tiss Org Cult 33:237-46). PLB preparations can be obtained using methods as described in Example XX. See, also, Mudalige, 2003, Id.; Lee et al., 2013, Am J Bot. 100(11):2121-31; and Chen et al., 2002, In Vitro Cellular & Developmental Biology 38(5):441-5.

### EXAMPLES

The Examples that follow are illustrative of specific embodiments of the invention, and various uses thereof. They are set forth for explanatory purposes only, and are not to be taken as limiting the invention.

### Example 1

### Isolation of Dendrobium Flavonoid 3′-Hydroxylase

Inflorescences of Dendrobium Jaquelyn Thomas ‘Uniwai Prince’ (UH 503) were harvested from University of Dubuque greenhouse grown plants. Total RNA was extracted from unopened buds according to the method of Champagne & Kuehnle, 2000, “Lindleyana 15:165-8.

cDNA was synthesized from 5 μg of total RNA using 200 units of SuperScript III reverse transcriptase (Invitrogen) according to conventional methods. Oligo dT (dT16 or dT20-T7) primers were used for first strand cDNA synthesis. The reaction was stopped by incubation of the mixture at 70° C. for 15 min. The RNA template was removed by incubating the reaction mixture with 2 units of RNase H (Promega) at 37° C. for 20 min. Resultant cDNA strands were used as the template for RT-PCR with degenerate primers targeted to the specific conserved regions of F3′H amino acid sequence alignment of publicly available monocot and some dicot sequences. (Arabidopsis thaliana: AF271651, Oryza sativa: ACO21892, Pelargonium x hortorum: AF315465, Petunia hybrida: AF155332, Torenia hybrida: AB0057673, and Sorghum bibolor. AY675075, and Zea mays: HQ699781).

Two degenerate primers, Den-degen-F3′H-for GGNGTNGAYGTNAARGG (SEQ ID NO:3) and Den-F3′H-Rev CCRTANGCYTCYTCCAT (SEQ ID NO:4), were used at a 1.20 μM final concentration in a 25 μL PCR reaction. Initial denaturation was done at 95° C. for 2 min followed by 30 cycles of amplification at 94° C. for 30 s, 49° C. for 30 s and 68° C. for 30 s. A final extension was carried out at 68° C. for 7 min. The resultant products were separated on a 1.5% agarose gel in 1×TAE electrophoresis buffer. A gel fragment containing a 180 base pair band was excised and cleaned using Qiagen MinElute Gel extraction kit and was cloned into a pGEM-T easy vector system according to conventional methods and the supplier's instructions.

A partial sequence of the putative Dendrobium F3′H was determined by sequencing cloned cDNA with T7 and Sp6 primers. The remainder of the F3′H gene was isolated using 5′ and 3′ RACE (Rapid Amplification of cDNA ends). 3′RACE was performed using this same cDNA with a gene-specific forward primer ATGACGGCGACGTTGATTCATG (SEQ ID NO:5) and T7 primer TAATACGACTCACTATAGGG (SEQ ID NO:6) at a 10:1 concentration ratio. Amplification for 35 cycles was performed under amplification conditions comprising 94° C. for 30 s, 55° C. for 30 s, and 68° C. for 30 s followed by a final extension at 68° C. for 7 min. Resultant PCR products were gel purified, cloned into pGEM-T easy vector and sequenced as described above.

For 5′RACE this same RNA was used with a 5′RACE kit from Invitogen. Three primers were designed from the isolated partial clone sequence. Den-F3′H-end primer, TTAAACATCTTTAGGATATGC (SEQ ID NO:7) was used as the gene specific primer to synthesize the first strand using SuperScript III reverse transcriptase enzyme. Primary PCR was performed for 30 cycles using Den-F3′H-12 primer GAGCCCATAAGCCTCTTCCAT (SEQ ID NO:8) at 94° C. for 30 s, 55° C. for 30 s, and 68° C. for 1.40 min. Primary PCR product was diluted 1:10 in sterile water. Diluted primary PCR product was used as the template to carry out secondary PCR. Nested PCR was carried out with primer Den-F3′H-11 GATTCTTCGCCCAGCGCCGAACGG (SEQ ID NO:9) at 94° C. for 30 s, 55° C. for 30 s, and 68° C. for 1.30 min. Resultant PCR product was gel purified and inserted into a pGEM-T easy vector system as described above. Amplified DNA comprising full length F3′H-encoding sequence was cloned according to the 5′ and 3′ RACE sequences by PCR amplification with the Den-F3′H-start ATGGGCTTCATTTTCCTCTTTG (SEQ ID NO:10) and Den F3′H-end TTAAACATCTTTAGGATATGC (SEQ ID NO:11) primers. PCR amplification for 30 cycles was carried out at 94° C. for 30 s, 55° C. for 30 s, and 68° C. for 1.40 min. Resultant PCR product comprising a F3′H-encoding complete open reading frame was cloned into pGEM-T easy vector for further manipulations.

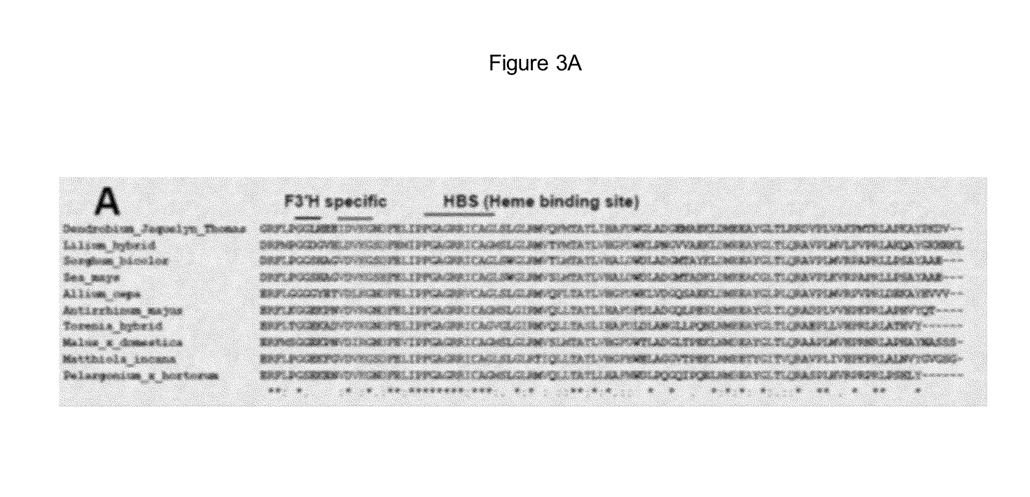

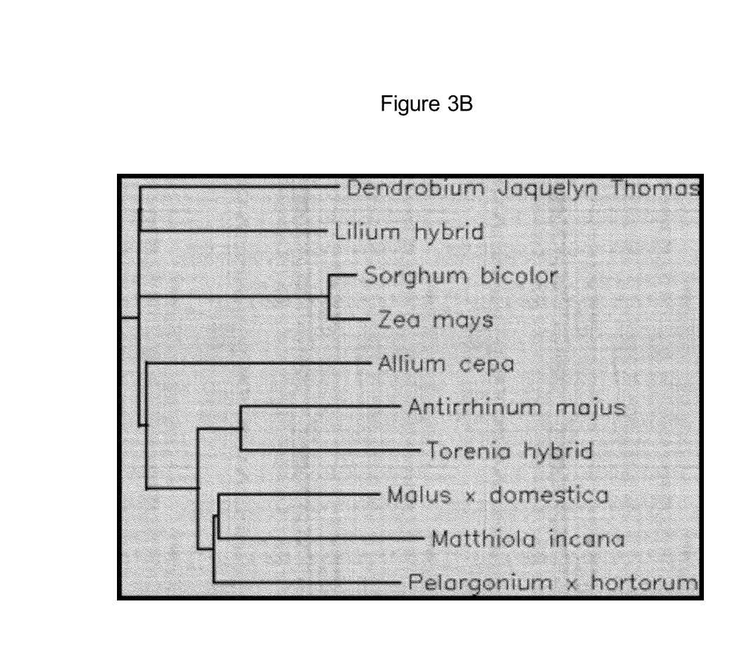

Dendrobium F3′H from Dendrobium orchid is 77% similar and 66% identical to the closest F3′H sequence found in GenBank (FIG. 3). Signature sequences that are specific to F3′H are conserved in DenF3′H. Amino acid sequence analysis suggests that it is most closely related to Lilioid monocots, followed by other grass monocots.

### Example 2

### Expression Profiles in Dendrobium

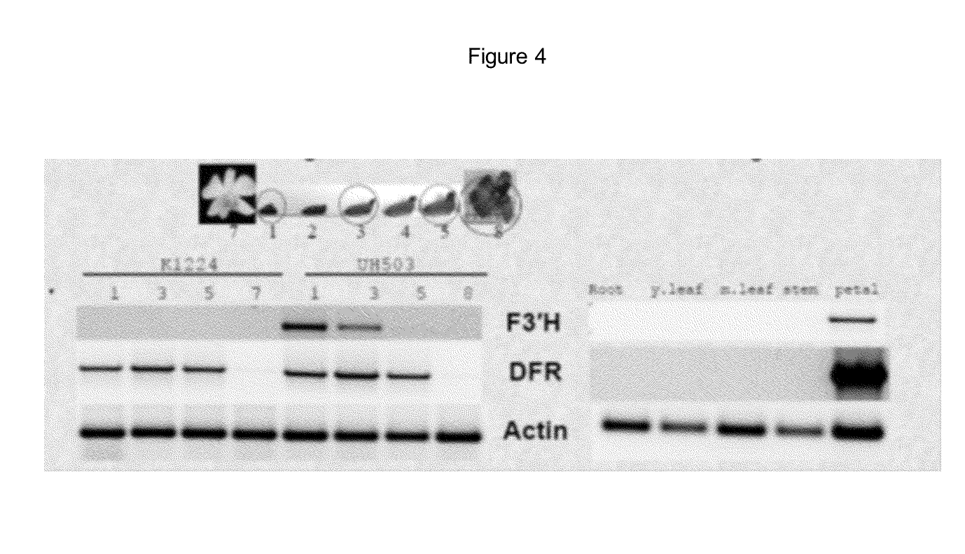

The temporal expression profile for F3′H from Dendrobium was determined for different stages of flower buds and spatial expression profile was determined for different plant organs. Thin layer chromatography of petals was performed according to the method of Kuehnle et al., 1997, Id. and Irani & Grotewald, 2005, BMC Plant Biol. 5:7. The results are shown in FIG. 4.

RT-PCR were performed using total RNA extracted from different plant organs (structures) to determine spatial expression profile while temporal expression profile of F3′H was assessed using RNA extracted from different floral bud stages. Actin was used to normalize RNA loading levels.

As shown in Obsuwan et al., 2007, Id., heterologous expression of Dendrobium—DFR in a mutant Petunia host indicated that the Dendrobium-DFR is capable of accepting DHK as a substrate to produce orange pelargonidin.

Qualitative expression analyses of F3′H by RT-PCR demonstrates that pelargonidin-accumulating mutants such as K1224 does not express F3′H. Therefore, the absence of competing enzyme, F3′H, is a prerequisite to convert DHK to orange pelargonidin via the activity of DFR in Dendrobium orchids.

### Example 3

### Transfection Procedures and Production of Transformed Orchid

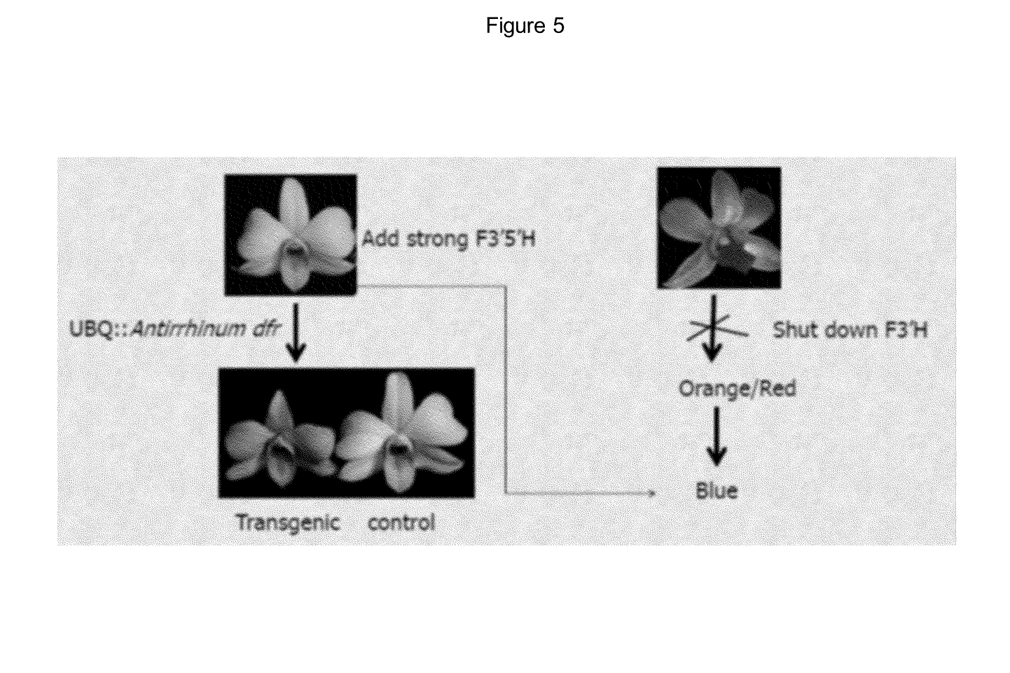

Dendrobium flower color can be modified through suppression of F3′H enzyme activity using sense and antisense suppression strategies (FIG. 5). To generate transgenic plants a Particle Inflow Gun can be used to deliver gold and/or tungsten particles carrying a recombinant genetic construct as set forth herein. (Finer et al., 1992, Plant Cell Reports 11:232-8; Vain et al., 1993, Plant Cell Tiss Org Cult 33:237-46).

Briefly, in one example, cell transformation procedure using the Particle Inflow Gun was carried out as follows:

(a) Sterilization of particles. 1. 50 mg of either tungsten or gold particles were suspended in 500 μL of 95% ethanol (prepared from 100% ethanol) and let set for 15 min. 2. The suspension was pun gently to pellet the particles and remove the supernatant. Pelleted particles werewashed with 500 μL sterile dH₂O 3 times. 3. The pellet was re-suspended in 330 μL sterile dH₂O to a final concentration of approximately 0.15 mg/μL.

(b) Precipitation of DNA upon the particles. 1. 5-15 μg of DNA construct (as described above) were precipitated upon 2.25 mg of 0.7-μm diameter tungsten (M10, 0.7-μm diameter on average; Sigma) or 1-μm diameter gold particles (Bio-Rad Laboratories). 2. An appropriate amount of sterilized particles (15 μL in my case) was removed and placed in a sterile eppendorf tube. 3. The appropriate DNA(s) were added in a total volume of 15 μL and mixed well. For control experiments, dH₂O was substituted for the DNA solution. For cotransformation experiments an additional 10-15 μg of a second plasmid DNA were added as appropriate. 4. 25 μL of 2.5 M CaCl₂ was added to the mixture and mixed well followed by addition of 10 μL of 100 mM spermidine (prepared fresh from 1M stock). The resulting solution was mixed well. 6. After the addition of spermidine, the solution was incubated on ice for 5 min, during which time the particles settled. 7. The top 45 μL were carefully removed and a 10 μL aliquot of the pellet was removed and placed on top of the filter mesh of either a 13-mm Swinney (Gelman Laboratory) or Swinnex (Millipore) filter. The filter was screwed into a Leur-lock attachment connected to the centered collar (see bombardment procedure below).

(c) Preparation of PLBs. 1. PLBs are made by sawing seeds in a liquid MS media supplemented with 15% coconut water and 3% sucrose and growing them on a shaker (100 rpm) with light. 2. Once the PLBs are 0.5 cm in diameter, they are placed on MS media plates and bombarded as described below. (See Mudalige, 2003, Id.).

(d) Bombardment procedure. 1. The top 45 μL of the precipitation mixture (see above) was carefully removed and a 10 μL aliquot of the pellet was placed on top of the filter mesh of either a 13-mm Swinney (Gelman Laboratory) or Swinnex (Millipore) filter. 2. The filter was screwed into a Leur-lock attachment connected to the centered collar. 3. The Petri dish top from the PLB tissue preparation above was removed and the bottom placed upon the stand. 4. The plexiglass door was attached, screwed tight, and a vacuum pulled to between 25-30 mm Hg. 5. A 50-ms burst of pressurized helium gas was released into the chamber through the filter unit by the action of the timer relay-driven solenoid (there will be a splash). 6. The vacuum was gently broken and the cell suspension was diluted in 6 mL of media. 7. Cells were grown for three days without selection at 28° C. in a humidity chamber, which is a sealed plastic-ware container with damp paper towels lining the bottom. 8. Over the next three days the culture was expanded to 10 mL by the daily addition of 1 mL of media. 9. After three days, the cells were counted and freshly prepared paromomycin added to a final concentration of 20-50 μg/mL (determined empirically). 10. Cells were grown for 2 days at 28° C. before assessment of transformation efficiency.

### Example 4

### Shutting Down of F3′H Gene Expression in Dendrobium Buds and Dendrobium plant

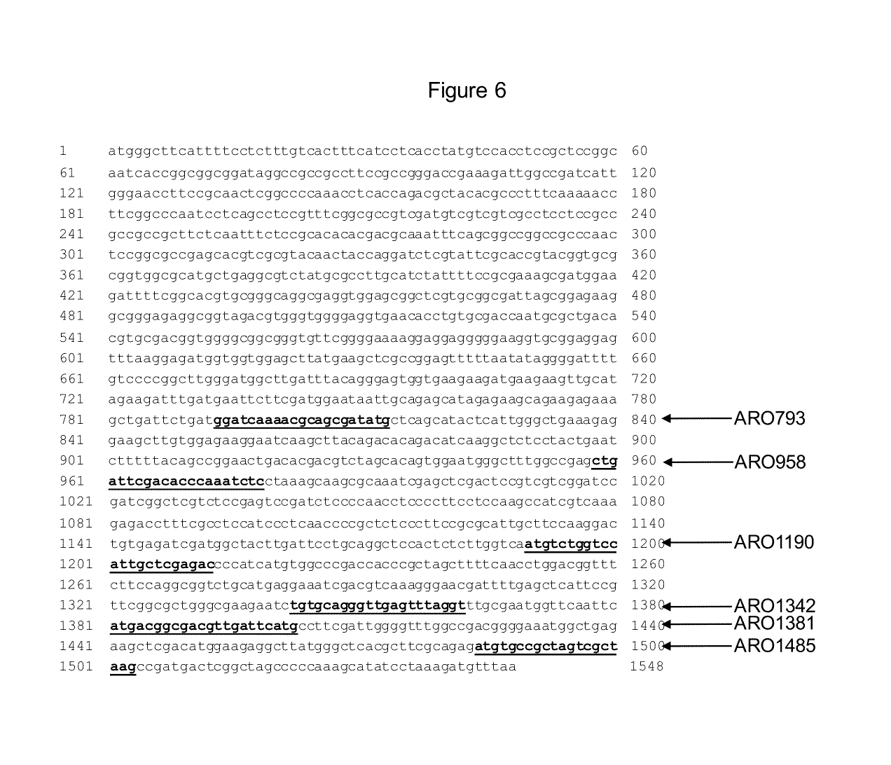

The Web microRNA Designer (WMD) was used as a platform for automated artificial microRNA (amiRNA) design against the F3′H gene. See Schwab et al., Plant Cell. 18(5):1121-33. Since orchids do not have a fully sequenced genome, Zea mays and Oryza sativa genomes were used as the reference genome to avoid silencing of genes other than the F3′H gene due to sequence similarity. The selected ARO sequences designed using WMD software are shown in Table 1, and the annealing regions of each ARO to the F3′H sequence are shown in FIG. 6. The ARO sequences in Table 1 were chosen because each of them individually targeted a unique region of the F3′H gene and each was closest to its particular target sequence. Both the 5′ and 3′ ends of the ARO molecules were modified by methylation to avoid degradation by exonucleases. The ARO sequences were synthesized by Integrated DNA Technologies.

TABLE 1
ARO molecules designed to shut
down F3′H in Dendrobium buds.

| | | | Hybrid- |
|---|---|---|---|
| | | Hybrid- | ization |
| | | ization | energy |
| | | energy | of ARO |
| | | of ARO to | to target |
| | | perfectly- | site in |
| | | matching | F3′H |
| ARO | | complement | gene |
| Molecules | Sequence | (kcal/mol) | (kcal/mol) |
| ARO793 | TATCGCTGCGT | -43.93 | -37.45 |
| (SEQ ID NO: 22) | TTTGATGCGT | | |
| ARO1190 | TTCGAGCAATG | -46.00 | -46.03 |
| (SEQ ID NO: 23) | GACCAGACAT | | |
| ARO958 | TAGATTTGGGT | -42.83 | -41.42 |
| (SEQ ID NO: 24) | GTCGAATCAG | | |
| ARO1342 | TCTAAACTCAA | -47.15 | -36.44 |
| (SEQ ID NO: 25) | CCCTCCACGG | | |
| ARO1381 | TTGAATCAACG | -44.15 | -43.84 |
| (SEQ ID NO: 26) | TCGCCGTCAT | | |
| ARO1485 | TATCGGCTTAG | -43.84 | -43.28 |
| (SEQ ID NO: 27) | CGACCAGCGG | | |

Purple-colored Dendrobium flower buds were used to test the direct delivery of each of the ARO molecules. The small and medium buds were utilized because they have the highest expression of anthocyanin biosynthetic genes (Mudalige et al. 2012, Id.). Intact flower buds, cut inflorescences, and surface sterilized excised individual buds were used to test the efficiency of ARO uptake (FIG. 7).

Several methods of ARO delivery were tested. First, each ARO molecule was individually fed via the cut end of the inflorescence and through the pedicel of individual buds, similar to the procedure used by Sun et al., 2005, Plant J. 44:128-38. Second, Unnamalai et al., 2004, FEBS Lett. 566:307-10) demonstrated that attaching the amiRNA to a short peptide molecule known as the protein transduction domain (PTD) increased the uptake of amiRNA by a significant amount. Similarly, a polyarginine PTD (SEQ ID NO:92) was attached to each ARO molecule, and the resultant complex was transported into flower buds through the xylem along with the uptake of water. Third, a concentrated solution of ARO or ARO+PTD complex was also injected directly into the flower buds via the pedicel. The smallest possible needle was used to reduce any damage due to wounding. Fourth, young flower buds were excised and surface sterilized in 3% chlorox solution for 5 min followed by three washes in sterile water. Cleaned buds were placed on Murashigae and Skoog media (MS media; PhytoTechnology Laboratories) supplemented with sucrose and Gamborg vitamins. A varying concentration of ARO from 1 ng/mL to 100 ng/mL was added to the media to find the most effective concentration for gene silencing. Direct feeding through the petiole for 2-3 days was found to yield the most effective reduction in F3′H expression.

Total RNA was isolated from control and ARO treated samples before and after the treatments using procedure described in Champagne & Kuehnle, 2000, Lindleyana 15:165-8. 5 μg of total RNA was converted to complementary DNA (cDNA) using reverse transcriptase III enzyme (Life Technologies). Resultant cDNA was used to amplify the remaining F3′H transcripts with oligonucleotide primers spanning the ARO-guided cleavage site. Sterile water was used as the negative control for feeding, and the actin gene was used as a loading control. Based on the RT-PCR results shown in FIG. 8, ARO958 (SEQ ID NO:24) and ARO1381 (SEQ ID NO:26) most effectively reduced F3′H expression. ARO793 (SEQ ID NO:22), ARO1190 (SEQ ID NO:23), and ARO1342 (SEQ ID NO:25) also lowered F3′H expression. Based on these results, the following regions of the F3′H sequence were identified for use construction of antisense gene constructs for rerouting the anthocyanin biosynthesis: nucleotides 793-1506, nucleotides 958-1506, nucleotides 793-1402, and/or nucleotides 958-1402. These antisense construct sequences are set forth in SEQ ID NOs:93-96, as shown in Table 2.

TABLE 2
Antisense constructs for insertion into Dendrobium PLBs.

| Antisense Construct | Sequence |
|---|---|
| Antisense construct corresponding to | SEQ ID NO: 93 |
| nucleotides 793-1506 of F3′H | |
| Antisense construct corresponding to | SEQ ID NO: 94 |
| nucleotides 958-1506 of F3′H | |
| Antisense construct corresponding to | SEQ ID NO: 95 |
| nucleotides 793-1402 of F3′H | |
| Antisense construct corresponding to | SEQ ID NO: 96 |
| nucleotides 958-1402 of F3′H | |

Each antisense construct of Table 2 was amplified by PCR and cloned into PBI121 under the CaMV 35S promoter. Clones were inserted into the PLBs of a dark purple Dendrobium hybrid to shut down the F3′H activity and reroute the anthocyanin biosynthesis towards orange pelargonidin and/or blue delphinidin with using the method below, as previously performed in Mudalige, 2003, Id.).

A Dendrobium hybrid with dark purple flowers was self-pollinated and kept in the greenhouse for 4.5 months until the seed capsule matured. Mature green seed capsules prior to splitting were harvested and surface sterilized by immersion in 70% ethanol followed by brief flaming. Mature seeds were removed using a sterile scalpel and a spatula. Seeds were placed in commercial liquid growth media (Vacin and Went media, Phytotechnology Labs) supplemented with 3% sucrose (w/v) and 15% coconut water (v/v) (phytotech labs) for the development of protocorms (tuber shaped undifferentiated young seedlings). Protocorms were multiplied to generate undifferentiated tissues with PLBs by maintaining the tissues in the same media with shaking at 100 rpm, 16 h photoperiod of 19.0 μmol m⁻² sec⁻¹ provided by cool white and Gro-Lux fluorescent lamps (GTE Corps). 46 PLBs (½-1 cm diameter) were placed on ½ strength MS media supplemented with 2% sucrose and 0.7% granulated phyto agar in 6.0×1.5 cm disposable sterile petri plates (Fisher Scientific). They were kept overnight in a dark drawer and bombarded with 1.0 μM diameter gold particles with precipitated plasmid DNA using Particle Inflow Gun (Davies, 2013, Id.).

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

What is claimed is:

1. A method for producing a transgenic plant, comprising:
(a) transfecting a plant with a genetic construct comprising an antisense suppressor of a nucleic acid molecule having at least 90% identity to a nucleotide sequence set forth in SEQ ID NO:1; and
(b) expressing the genetic construct in cells of the plant.

2. The method of claim 1, wherein the antisense suppressor comprises an antisense suppressor having at least 90% identity to a sequence set forth in any one of SEQ ID NOs:93-96.

3. The method of claim 1, wherein the genetic construct is expressed in the protocorm-like bodies of the plant.

4. The method of claim 1, wherein the transgenic plant is a flower color-changed plant and wherein the plant is a native-color plant.

5. The method of claim 4, wherein the flower color-changed plant and the native-color plant are of the Orchidaceae family.

6. The method of claim 5, wherein the flower color-changed plant and the native-color plant are Dendrobium orchids.

7. A method for producing a flower color-changed plant having an orange flower, comprising:
(a) transfecting a native-color plant having a purple flower with a genetic construct comprising an antisense suppressor of a nucleic acid molecule having at least 90% identity to a nucleotide sequence set forth in SEQ ID NO:1; and
(b) expressing the genetic construct in cells of the plant.

8. The method of claim 7, wherein the antisense suppressor comprises an antisense suppressor having at least 90% identity to a sequence set forth in any one of SEQ ID NOs:93-96.

9. The method of claim 7, wherein the genetic construct is expressed in the protocorm-like bodies of the plant.

10. The method of claim 7, wherein the flower color-changed plant and the native-color plant are of the Orchidaceae family.

11. The method of claim 10, wherein the flower color-changed plant and the native-color plant are Dendrobium orchids.

12. A flower color-changed plant produced by the method of claim 1.

13. The flower color-changed plant according to claim 12, wherein the flower color-changed plant is of the Orchidaceae family.

14. The flower color-changed plant according to claim 13, wherein the flower color-changed plant is a Dendrobium orchid.

15. A flower color-changed plant comprising in cells thereof a genetic construct comprising an antisense suppressor of a nucleic acid molecule having at least 90% identity to a nucleotide sequence set forth in SEQ ID NO:1.

16. The flower color-changed plant of claim 15, wherein the antisense suppressor comprises an antisense suppressor having at least 90% identity to a sequence set forth in any one of SEQ ID NOs:93-96.

17. The flower color-changed plant of claim 15, wherein the flower color-changed plant is of the Orchidaceae family.

18. The flower color-changed plant of claim 17, wherein the flower color-changed plant is a Dendrobium orchid.


SEQUENCE LISTING (96 sequences)

SEQ ID NO: 1 - DNA, 1548 bp, Dendrobium  Jaquelyn Thomas ′Uniwai Prince′
  CDS (1)..(1548)
atgggcttcattttcctctttgtcactttcatcctcacctatgtccacctccgctccggcaatcaccggcggcggataggccgccgccttccgccgggaccgaaagattggccgatcattgggaaccttccgcaactcggccccaaacctcaccagacgctacacgccctttcaaaaaccttcggcccaatcctcagcctccgtttcggcgccgtcgatgtcgtcgtcgcctcctccgccgccgccgcttctcaatttctccgcacacacgacgcaaatttcagcggccggccgcccaactccggcgccgagcacgtcgcgtacaactaccaggatctcgtattcgcaccgtacggtgcgcggtggcgcatgctgaggcgtctatgcgccttgcatctattttccgcgaaagcgatggaagattttcggcacgtgcgggcaggcgaggtggagcggctcgtgcggcgattagcggagaaggcgggagaggcggtagacgtgggtggggaggtgaacacctgtgcgaccaatgcgctgacacgtgcgacggtggggcggcgggtgttcggggaaaaggaggagggggaaggtgcggaggagtttaaggagatggtggtggagcttatgaagctcgccggagtttttaatataggggattttgtccccggcttgggatggcttgatttacagggagtggtgaagaagatgaagaagttgcatagaagatttgatgaattcttcgatggaataattgcagagcatagagaagcagaagagaaagctgattctgatggatcaaaacgcagcgatatgctcagcatactcattgggctgaaagaggaagcttgtggagaaggaatcaagcttacagacacagacatcaaggctctcctactgaatctttttacagccggaactgacacgacgtctagcacagtggaatgggctttggccgagctgattcgacacccaaatctcctaaagcaagcgcaaatcgagctcgactccgtcgtcggatccgatcggctcgtctccgagtccgatctccccaacctccccttcctccaagccatcgtcaaagagacctttcgcctccatccctcaaccccgctctcccttccgcgcattgcttccaaggactgtgagatcgatggctacttgattcctgcaggctccactctcttggtcaatgtctggtccattgctcgagaccccatcatgtggcccgaccacccgctagcttttcaacctggacggtttcttccaggcggtctgcatgaggaaatcgacgtcaaagggaacgattttgagctcattccgttcggcgctgggcgaagaatctgtgcagggttgagtttaggtttgcgaatggttcaattcatgacggcgacgttgattcatgccttcgattggggtttggccgacggggaaatggctgagaagctcgacatggaagaggcttatgggctcacgcttcgcagagatgtgccgctagtcgctaagccgatgactcggctagcccccaaagcatatcctaaagatgtttaa

SEQ ID NO: 2 - protein, 515 aa, Dendrobium  Jaquelyn Thomas ′Uniwai Prince′
MGFIFLFVTFILTYVHLRSGNHRRRIGRRLPPGPKDWPIIGNLPQLGPKPHQTLHALSKTFGPILSLRFGAVDVVVASSAAAASQFLRTHDANFSGRPPNSGAEHVAYNYQDLVFAPYGARWRMLRRLCALHLFSAKAMEDFRHVRAGEVERLVRRLAEKAGEAVDVGGEVNTCATNALTRATVGRRVFGEKEEGEGAEEFKEMVVELMKLAGVFNIGDFVPGLGWLDLQGVVKKMKKLHRRFDEFFDGIIAEHREAEEKADSDGSKRSDMLSILIGLKEEACGEGIKLTDTDIKALLLNLFTAGTDTTSSTVEWALAELIRHPNLLKQAQIELDSVVGSDRLVSESDLPNLPFLQAIVKETFRLHPSTPLSLPRIASKDCEIDGYLIPAGSTLLVNVWSIARDPIMWPDHPLAFQPGRFLPGGLHEEIDVKGNDFELIPFGAGRRICAGLSLGLRMVQFMTATLIHAFDWGLADGEMAEKLDMEEAYGLTLRRDVPLVAKPMTRLAPKAYPKDV

SEQ ID NO: 3 - DNA, 17 bp, Artificial Sequence
  Synthetic oligonucleotide
ggngtngaygtnaargg

SEQ ID NO: 4 - DNA, 17 bp, Artificial Sequence
  Synthetic oligonucleotide
ccrtangcytcytccat

SEQ ID NO: 5 - DNA, 22 bp, Artificial Sequence
  Synthetic oligonucleotide
atgacggcgacgttgattcatg

SEQ ID NO: 6 - DNA, 20 bp, Artificial Sequence
  Synthetic oligonucleotide
taatacgactcactataggg

SEQ ID NO: 7 - DNA, 21 bp, Artificial Sequence
  Synthetic oligonucleotide
ttaaacatctttaggatatgc

SEQ ID NO: 8 - DNA, 21 bp, Artificial Sequence
  Synthetic oligonucleotide
gagcccataagcctcttccat

SEQ ID NO: 9 - DNA, 24 bp, Artificial Sequence
  Synthetic oligonucleotide
gattcttcgcccagcgccgaacgg

SEQ ID NO: 10 - DNA, 22 bp, Artificial Sequence
  Synthetic oligonucleotide
atgggcttcattttcctctttg

SEQ ID NO: 11 - DNA, 21 bp, Artificial Sequence
  Synthetic oligonucleotide
ttaaacatctttaggatatgc

SEQ ID NO: 12 - protein, 98 aa, Dendrobium  Jaquelyn Thomas
GRFLPGGLHEEIDVKGNDFELIPFGAGRRICAGLSLGLRMVQFMTATLIHAFDWGLADGEMAEKLDMEEAYGLTLRRDVPLVAKPMTRLAPKAYPKDV

SEQ ID NO: 13 - protein, 100 aa, Lilium hybrid
DRFMPGGDGVHLDVKGSDFEMIPFGAGRRICAGMSLGLRMVTFMTATLVHGFDWKLPNGVVAEKLDMEEAYGLTLQRAVPLMVLPVPRLAKQAYGKHEKL

SEQ ID NO: 14 - protein, 97 aa, Sorghum bicolor
DRFLPGGSHAGVDVKGSDFELIPFGAGRRICAGLSWGLRMVTLMTATLVHALDWDLADGMTAYKLDMEEAYGLTLQRAVPLMVRPAPRLLPSAYAAE

SEQ ID NO: 15 - protein, 97 aa, Zea mays
DRFLPGGSHAGVDVKGSEFELIPFGAGRRICAGLSWGLRMVSLMTATLVHALDWDLADGMTADKLDMEEACGLTLQRAVPLKVRPAPRLLPSAYAAE

SEQ ID NO: 16 - protein, 98 aa, Allium cepa
ERFLGGGGYETVDLKGNDFELIPFGAGRRVCAGLSLGLRMVQFLTATLVHGFDWKLVDGQSAEKLDMEEAYGLPLQRAVPLMVRPVPRLDEKAYHVVV

SEQ ID NO: 17 - protein, 96 aa, Antirrhinum majus
ERFLKGGEKPNVDVRGNDFELIPFGAGRRICAGMSLGIRMVQLLTATLIHAFDFDLADGQLPESLNMEEAYGLTLQRADPLVVHPKPRLAPHVYQT

SEQ ID NO: 18 - protein, 94 aa, Torenia hybrid
ERFLTGGEKADVDVKGNDFELIPFGAGRRICAGVGLGIRMVQLLTASLIHAFDLDLANGLLPQNLNMEEAYGLTLQRAEPLLVHPRLRLATHVY

SEQ ID NO: 19 - protein, 99 aa, Malus domestica
ERFMSGGEKPNVDIRGNDFEVIPFGAGRRICAGMSLGLRMVSLMTATLVHGFDWTLADGLTPEKLNMDEAYGLTLQRAAPLMVHPRNRLAPHAYNASSS

SEQ ID NO: 20 - protein, 99 aa, Matthiola incana
ERFLPGGEKFGVDVKGSDFELIPFGAGRRICAGLSLGLRTIQLLTATLVHGFEWELAGGVTPEKLNMEETYGITVQRAVPLIVHPKPRLALNVYGVGSG

SEQ ID NO: 21 - protein, 94 aa, Pelargonium hortorum
ERFLPGSEKENVDVKGNDFELIPFGAGRRICAGMSLGLRMVQLLTATLLHAFNWDLPQGQIPQELNMDEAYGLTLQRASPLHVRPRPRLPSHLY

SEQ ID NO: 22 - DNA, 23 bp, Artificial Sequence
  ARO793
tatcgctgcgttttgatgcgtar

SEQ ID NO: 23 - DNA, 21 bp, Artificial Sequence
  ARO1190
ttcgagcaatggaccagacat

SEQ ID NO: 24 - DNA, 21 bp, Artificial Sequence
  ARO958
tagatttgggtgtcgaatcag

SEQ ID NO: 25 - DNA, 21 bp, Artificial Sequence
  ARO1342
tctaaactcaaccctccacgg

SEQ ID NO: 26 - DNA, 21 bp, Artificial Sequence
  ARO1381
ttgaatcaacgtcgccgtcat

SEQ ID NO: 27 - DNA, 21 bp, Artificial Sequence
  ARO1485
tatcggcttagcgaccagcgg

SEQ ID NO: 28 - DNA, 21 bp, Artificial Sequence
  ARO molecule
ttatcgctgcgttatggtcca

SEQ ID NO: 29 - DNA, 21 bp, Artificial Sequence
  ARO molecule
tatcgctgcgtttagatgcat

SEQ ID NO: 30 - DNA, 21 bp, Artificial Sequence
  ARO molecule
tcttagcgactagtgccacat

SEQ ID NO: 31 - DNA, 21 bp, Artificial Sequence
  ARO molecule
tatcgctgcgtttagacccat

SEQ ID NO: 32 - DNA, 21 bp, Artificial Sequence
  ARO molecule
tctaaactcaaccctgcacgg

SEQ ID NO: 33 - DNA, 21 bp, Artificial Sequence
  ARO molecule
taaggcatgaatcaccgtctc

SEQ ID NO: 34 - DNA, 21 bp, Artificial Sequence
  ARO molecule
tctaaactcaaccctgcgcag

SEQ ID NO: 35 - DNA, 21 bp, Artificial Sequence
  ARO molecule
tctaaactcaaccgtgcgcag

SEQ ID NO: 36 - DNA, 21 bp, Artificial Sequence
  ARO molecule
ttcgagcaatggaccacacat

SEQ ID NO: 37 - DNA, 21 bp, Artificial Sequence
  ARO molecule
ttatcgctgcgttttgagcct

SEQ ID NO: 38 - DNA, 21 bp, Artificial Sequence
  ARO molecule
tatcggcttagcgagtaccgg

SEQ ID NO: 39 - DNA, 21 bp, Artificial Sequence
  ARO molecule
tcttagcgactagcggcacat

SEQ ID NO: 40 - DNA, 21 bp, Artificial Sequence
  ARO molecule
tcttagcgactagtggcccat

SEQ ID NO: 41 - DNA, 21 bp, Artificial Sequence
  ARO molecule
tctaaactcaaccctggacgg

SEQ ID NO: 42 - DNA, 21 bp, Artificial Sequence
  ARO molecule
tcttagcgactagccgcacat

SEQ ID NO: 43 - DNA, 21 bp, Artificial Sequence
  ARO molecule
ttcgagcaatggaccagacac

SEQ ID NO: 44 - DNA, 21 bp, Artificial Sequence
  ARO molecule
ttatcgctgcgttttgagcca

SEQ ID NO: 45 - DNA, 21 bp, Artificial Sequence
  ARO molecule
ttcgagcaatggaccacacac

SEQ ID NO: 46 - DNA, 21 bp, Artificial Sequence
  ARO molecule
ttgaatcaacgtcggcctcat

SEQ ID NO: 47 - DNA, 21 bp, Artificial Sequence
  ARO molecule
ttgaatcaacgtcggcgtctt

SEQ ID NO: 48 - DNA, 21 bp, Artificial Sequence
  ARO molecule
tctaaactcaaccctccgcag

SEQ ID NO: 49 - DNA, 21 bp, Artificial Sequence
  ARO molecule
ttatcgctgcgttttcatcct

SEQ ID NO: 50 - DNA, 21 bp, Artificial Sequence
  ARO molecule
tagtcatcggctttgcgccta

SEQ ID NO: 51 - DNA, 21 bp, Artificial Sequence
  ARO molecule
tctaaactcaacccggcgcag

SEQ ID NO: 52 - DNA, 21 bp, Artificial Sequence
  ARO molecule
tctaaactcaaccccgcgcag

SEQ ID NO: 53 - DNA, 21 bp, Artificial Sequence
  ARO molecule
tctaaactcaacccggcacgg

SEQ ID NO: 54 - DNA, 21 bp, Artificial Sequence
  ARO molecule
tatcggcttagcgagtagcgg

SEQ ID NO: 55 - DNA, 21 bp, Artificial Sequence
  ARO molecule
tcttagcgactagggggacat

SEQ ID NO: 56 - DNA, 21 bp, Artificial Sequence
  ARO molecule
ttatcgctgcgttatgctcca

SEQ ID NO: 57 - DNA, 21 bp, Artificial Sequence
  ARO molecule
ttatcgctgcgttttcatcga

SEQ ID NO: 58 - DNA, 21 bp, Artificial Sequence
  ARO molecule
tcttagcgactagcgccactt

SEQ ID NO: 59 - DNA, 21 bp, Artificial Sequence
  ARO molecule
tcttagcgactagcgggacct

SEQ ID NO: 60 - DNA, 21 bp, Artificial Sequence
  ARO molecule
ttcgagcaatggaccacacag

SEQ ID NO: 61 - DNA, 21 bp, Artificial Sequence
  ARO molecule
ttgaatcaacgtcgccgtctt

SEQ ID NO: 62 - DNA, 21 bp, Artificial Sequence
  ARO molecule
tatcggcttagcgagtagcag

SEQ ID NO: 63 - DNA, 21 bp, Artificial Sequence
  ARO molecule
ttgaatcaacgtcggcgtcat

SEQ ID NO: 64 - DNA, 21 bp, Artificial Sequence
  ARO molecule
tagtcatcggcttaccgacta

SEQ ID NO: 65 - DNA, 21 bp, Artificial Sequence
  ARO molecule
tcttagcgactagcggcgcat

SEQ ID NO: 66 - DNA, 21 bp, Artificial Sequence
  ARO molecule
taaggcatgaatcaacggcgc

SEQ ID NO: 67 - DNA, 21 bp, Artificial Sequence
  ARO molecule
ttcgagcaatggaccacacct

SEQ ID NO: 68 - DNA, 21 bp, Artificial Sequence
  ARO molecule
tcttagcgactagcgccacac

SEQ ID NO: 69 - DNA, 21 bp, Artificial Sequence
  ARO molecule
tcttagcgactagccgcacac

SEQ ID NO: 70 - DNA, 21 bp, Artificial Sequence
  ARO molecule
ttatcgctgcgttttcagcca

SEQ ID NO: 71 - DNA, 21 bp, Artificial Sequence
  ARO molecule
ttgaatcaacgtcggcgtcac

SEQ ID NO: 72 - DNA, 21 bp, Artificial Sequence
  ARO molecule
ttcgagcaatggacgagacac

SEQ ID NO: 73 - DNA, 21 bp, Artificial Sequence
  ARO molecule
tatcggcttagcgaccagctg

SEQ ID NO: 74 - DNA, 21 bp, Artificial Sequence
  ARO molecule
tcttagcgactagccgcacct

SEQ ID NO: 75 - DNA, 21 bp, Artificial Sequence
  ARO molecule
tcttagcgactaggggcgcat

SEQ ID NO: 76 - DNA, 21 bp, Artificial Sequence
  ARO molecule
tcttagcgactagccgcgcat

SEQ ID NO: 77 - DNA, 21 bp, Artificial Sequence
  ARO molecule
ttatcgctgcgttttgctcct

SEQ ID NO: 78 - DNA, 21 bp, Artificial Sequence
  ARO molecule
tctaaactcaaccctgcccgg

SEQ ID NO: 79 - DNA, 21 bp, Artificial Sequence
  ARO molecule
tcttagcgactagcggcgcaa

SEQ ID NO: 80 - DNA, 21 bp, Artificial Sequence
  ARO molecule
ttatcgctgcgtttgggtcca

SEQ ID NO: 81 - DNA, 21 bp, Artificial Sequence
  ARO molecule
tatcggcttagcgcctagcgc

SEQ ID NO: 82 - DNA, 21 bp, Artificial Sequence
  ARO molecule
tatcaagccatcccaagccgg

SEQ ID NO: 83 - DNA, 21 bp, Artificial Sequence
  ARO molecule
tatcaagccatcccaaggctg

SEQ ID NO: 84 - DNA, 21 bp, Artificial Sequence
  ARO molecule
tatcggcttagcgactggccg

SEQ ID NO: 85 - DNA, 21 bp, Artificial Sequence
  ARO molecule
tatcggcttagcgaccaccgg

SEQ ID NO: 86 - DNA, 21 bp, Artificial Sequence
  ARO molecule
tatcaagccatcccgagccgg

SEQ ID NO: 87 - DNA, 21 bp, Artificial Sequence
  ARO molecule
taaggcatgaatcagcggcgc

SEQ ID NO: 88 - DNA, 21 bp, Artificial Sequence
  ARO molecule
ttcgagcaatggaccaggcac

SEQ ID NO: 89 - DNA, 21 bp, Artificial Sequence
  ARO molecule
tatcaagccatcccgaggcgg

SEQ ID NO: 90 - DNA, 21 bp, Artificial Sequence
  ARO molecule
tcttagcgactagcggccctt

SEQ ID NO: 91 - DNA, 21 bp, Artificial Sequence
  ARO molecule
tcttagcgactagcggcgcct

SEQ ID NO: 92 - protein, 12 aa, Artificial Sequence
  Protein transduction domain
RRRRRRRRRRRR

SEQ ID NO: 93 - DNA, 714 bp, Artificial Sequence
  Antisense construct
cggcttagcgactagcggcacatctctgcgaagcgtgagcccataagcctcttccatgtcgagcttctcagccatttccccgtcggccaaaccccaatcgaaggcatgaatcaacgtcgccgtcatgaattgaaccattcgcaaacctaaactcaaccctgcacagattcttcgcccagcgccgaacggaatgagctcaaaatcgttccctttgacgtcgatttcctcatgcagaccgcctggaagaaaccgtccaggttgaaaagctagcgggtggtcgggccacatgatggggtctcgagcaatggaccagacattgaccaagagagtggagcctgcaggaatcaagtagccatcgatctcacagtccttggaagcaatgcgcggaagggagagcggggttgagggatggaggcgaaaggtctctttgacgatggcttggaggaaggggaggttggggagatcggactcggagacgagccgatcggatccgacgacggagtcgagctcgatttgcgcttgctttaggagatttgggtgtcgaatcagctcggccaaagcccattccactgtgctagacgtcgtgtcagttccggctgtaaaaagattcagtaggagagccttgatgtctgtgtctgtaagcttgattccttctccacaagcttcctctttcagcccaatgagtatgctgagcatatcgctgcgttttgatcc

SEQ ID NO: 94 - DNA, 549 bp, Artificial Sequence
  Antisense construct
cggcttagcgactagcggcacatctctgcgaagcgtgagcccataagcctcttccatgtcgagcttctcagccatttccccgtcggccaaaccccaatcgaaggcatgaatcaacgtcgccgtcatgaattgaaccattcgcaaacctaaactcaaccctgcacagattcttcgcccagcgccgaacggaatgagctcaaaatcgttccctttgacgtcgatttcctcatgcagaccgcctggaagaaaccgtccaggttgaaaagctagcgggtggtcgggccacatgatggggtctcgagcaatggaccagacattgaccaagagagtggagcctgcaggaatcaagtagccatcgatctcacagtccttggaagcaatgcgcggaagggagagcggggttgagggatggaggcgaaaggtctctttgacgatggcttggaggaaggggaggttggggagatcggactcggagacgagccgatcggatccgacgacggagtcgagctcgatttgcgcttgctttaggagatttgggtgtcgaatcag

SEQ ID NO: 95 - DNA, 610 bp, Artificial Sequence
  Antisense construct
catgaatcaacgtcgccgtcatgaattgaaccattcgcaaacctaaactcaaccctgcacagattcttcgcccagcgccgaacggaatgagctcaaaatcgttccctttgacgtcgatttcctcatgcagaccgcctggaagaaaccgtccaggttgaaaagctagcgggtggtcgggccacatgatggggtctcgagcaatggaccagacattgaccaagagagtggagcctgcaggaatcaagtagccatcgatctcacagtccttggaagcaatgcgcggaagggagagcggggttgagggatggaggcgaaaggtctctttgacgatggcttggaggaaggggaggttggggagatcggactcggagacgagccgatcggatccgacgacggagtcgagctcgatttgcgcttgctttaggagatttgggtgtcgaatcagctcggccaaagcccattccactgtgctagacgtcgtgtcagttccggctgtaaaaagattcagtaggagagccttgatgtctgtgtctgtaagcttgattccttctccacaagcttcctctttcagcccaatgagtatgctgagcatatcgctgcgttttgatcc

SEQ ID NO: 96 - DNA, 445 bp, Artificial Sequence
  Antisense construct
catgaatcaacgtcgccgtcatgaattgaaccattcgcaaacctaaactcaaccctgcacagattcttcgcccagcgccgaacggaatgagctcaaaatcgttccctttgacgtcgatttcctcatgcagaccgcctggaagaaaccgtccaggttgaaaagctagcgggtggtcgggccacatgatggggtctcgagcaatggaccagacattgaccaagagagtggagcctgcaggaatcaagtagccatcgatctcacagtccttggaagcaatgcgcggaagggagagcggggttgagggatggaggcgaaaggtctctttgacgatggcttggaggaaggggaggttggggagatcggactcggagacgagccgatcggatccgacgacggagtcgagctcgatttgcgcttgctttaggagatttgggtgtcgaatcag